(19) United States
(12) Patent Application Publication
(10) Pub. No.: US 2009/0082755 A1
(43) Pub. Date: Mar. 26, 2009

(54) PRESSURE FEEDBACK UNIT FOR A CATHETER

(76) Inventors: Zoran Milijasevic, Bayview (AU); Neil L. Anderson, Roseville (AU)

Correspondence Address: MORRISON & FOERSTER LLP, 755 PAGE MILL RD, PALO ALTO, CA, 94304-1018, US

(21) Appl. No.: 12/158,662

(22) PCT Filed: Nov. 27, 2006
(86) PCT No.: PCT/AU2006/001790

§ 371 Date: Nov. 18, 2008

Related U.S. Application Data
(60) Provisional application No. 60/753,675, filed on Dec. 23, 2005.

(51) Int. Cl. A61M 25/00

(52) U.S. Cl. 604/523

(57) ABSTRACT

A pressure feedback unit (10) for a catheter includes a support (18) for supporting a proximal end of an electrode sheath of the catheter. The unit (10) further includes a connector (12) for connection to a component of the catheter, the connector (12) and the support (18) being displaceable relative to each other. An urging element (28) is interposed between the support (18) and the connector (12) for urging the support (18) and the connector (12) to a rest condition relative to each other.

### CROSS-REFERENCE TO RELATED APPLICATIONS

The present application claims priority from U.S. Provisional Patent Application No. 60/753,675 filed on 23 Dec. 2005, the contents of which are incorporated herein by reference.

### FIELD

This invention relates generally to the field of catheters and, more particularly, to a pressure feedback unit for a catheter for monitoring the pressure applied at a distal region of the catheter, in use.

### BACKGROUND

In the heat treatment of a biological site in a patient's body, it is necessary to apply pressure to a distal end of a catheter being used to effect treatment at the site. The pressure to be applied needs to be monitored to ensure that the pressure applied is not that great that it can cause perforations and damage to the tissue.

Further, in order to reach the site, the catheter is often introduced into the patient's vascular system at a region remote from the site to be treated. The catheter is introduced into the vascular system using an introducer and the introducer is steered through the vascular system to the site. There is also a risk of damage to the vascular system resulting from an excessive application of pressure while the introducer is being steered.

The Applicant has filed an International Patent Application for an electrical lead under International Patent Application Number PCT/AU01/01339 dated 19 Oct. 2001. The contents of that application are incorporated in this specification by reference. The electrical lead forming the subject matter of the International Patent Application is suitable as an electrode sheath of a catheter. Due to the construction of the electrical lead, the lumen is unimpeded and a steering device for the catheter is receivable in the lumen to steer the catheter. Such a construction is useful to be able to monitor the pressure applied by the catheter, in use.

### SUMMARY

According to a first aspect of the invention, there is provided a pressure feedback unit for a catheter, the unit including

a support for supporting a proximal end of an electrode sheath of the catheter;

a connector for connection to a component of the catheter, the connector and the support being displaceable relative to each other; and

an urging element interposed between the support and the connector for urging the support and the connector to a rest condition relative to each other.

The support may directly or indirectly support the electrode sheath. In the latter case, one or more intermediate elements may be interposed between the proximal end of the electrode sheath and the support. For example, the intermediate element may be a Y-connector to which a source of irrigation fluid is connectable to supply irrigation fluid to a distal end of the electrode sheath.

The unit may include a housing carrying the connector. In an embodiment, the connector may be supported at a proximal end of the housing and a distal end of the housing may define the support for the electrode sheath. The connector may be displaceably arranged relative to the housing. In another embodiment, the connector may be fixed to a proximal end of the housing and the support may comprise an attachment element displaceably arranged relative to the housing. The attachment element may be an attachment plate slidably supported in the housing.

The urging element may be a coil spring.

The unit may include a guide formation, in the form of a guide tube, co-axially arranged with respect to the support and the connector, the guide tube defining a passage through which a steering device of the catheter passes, in use. The coil spring may be arranged about the guide tube.

The component of the catheter may be a handle and the connector may be configured to engage a distal end of the handle. The connector may define a receiving formation for receiving a part of the handle. Further, the connector may include an electrical connector for electrical connection between electrode conductors of the electrode sheath and electrical members, such as electrical cabling, associated with the handle.

The unit may include an indicator for indicating applied pressure. The indicator may comprise a marker slidably arranged in a slot in the housing. Markings, indicative of the pressure applied, may be demarcated on the housing adjacent the slot.

The unit may include a measuring device for measuring the applied pressure, the measuring device being responsive to the urging element. In addition, the unit may include a discernible alarm connected to the measuring device. The discernible alarm may be an audio and/or visual enunciator. More particularly, the measuring device may be a load cell and, with knowledge of the spring force of the spring, an electronic output of the load cell may be representative of the pressure applied. Further, the load cell may be calibrated to take into account system friction thereby minimising the effect of the system friction.

According to a second aspect of the invention, there is provided a pressure feedback unit for a catheter, the unit including

a support for supporting a proximal end of an electrode sheath of the catheter;

a connector for connection to a component of the catheter; and

a pressure transducer interposed between the support and the connector, a force applied to the transducer by one of the connector and the support being monitored by the transducer to give an indication of pressure applied.

### BRIEF DESCRIPTION OF THE DRAWINGS

FIG. 1 shows a three dimensional view of a pressure feedback unit, in accordance with an embodiment of the invention, for a catheter;

FIG. 2 shows a sectional isometric view of the unit of FIG. 1;

FIG. 3 shows a sectional side view of the unit of FIG. 1;

FIG. 4 shows a sectional side view of a pressure feedback unit, in accordance with another embodiment of the invention, for a catheter;

FIG. 5 shows a sectional isometric view of the unit of FIG. 4;

FIG. 6 shows an isometric view of the unit of FIG. 4;

FIG. 7 shows a plan view of a pressure feedback unit, in accordance with yet a further embodiment of the invention, for a catheter;

FIG. 8 shows a sectional side view of the unit of FIG. 7 in a first condition;

FIG. 9 shows a sectional side view of the unit of FIG. 7 in a second condition;

FIG. 10 shows a sectional, isometric view of the unit of FIG. 7 in its first condition;

FIG. 11 shows a sectional, isometric view of the unit of FIG. 7 in its second condition;

FIG. 12 shows a schematic, sectional side view of a distal part of a catheter, including a pressure feedback unit, in accordance with still a further embodiment of the invention, in a first condition; and

FIG. 13 shows a schematic, sectional side view of a distal part of the catheter of FIG. 12 with the unit in a second condition.

### DETAILED DESCRIPTION OF EXEMPLARY EMBODIMENTS

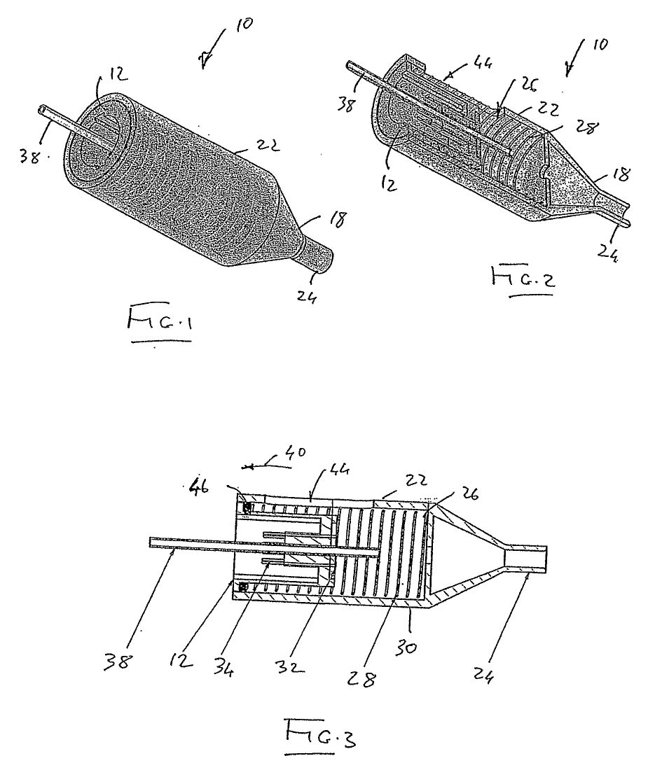

Referring initially to FIGS. 1 to 3 of the drawings, a first embodiment of a pressure feedback unit for a catheter is illustrated and is designated generally by the reference numeral 10. The unit 10 comprises a connector 12 for connection to a component, more particularly, a handle (not shown in this embodiment) of a catheter 16 (FIGS. 12 and 13 of the drawings).

In this embodiment, the unit 10 defines a support 18 for an electrode sheath (not shown in FIGS. 1 to 3 of the drawings but indicated at 20 in FIGS. 12 and 13 of the drawings). The support 18 supports a proximal end of the electrode sheath 20 and is defined by a distal end 24 of a housing 22. The distal end 24 of the housing 22 is in communication with an interior 26 of the housing 22. The connector 12 is arranged at a proximal end of the housing 22 and is slidably received in the interior 26 of the housing 22.

An urging element in the form of a coil spring 28 is received in the interior 26 of the housing 22 interposed between the connector 12 and the distal end 24 of the housing 22. The coil spring 28 has an outer diameter approximating the inner diameter of a cylindrical part 30 of the housing 22.

A distal end of the connector 12 carries a printed circuit board (PCB) 32 to which electrode conductors (not shown) of the electrode sheath 20 are connected. The PCB 32 is electrically connected to an electrical connector 34 which is connected to an appropriate connector (not shown) in the handle of the catheter 16 to make electrical connection between the electrode conductors of the electrode sheath 20 and, for example, a patient cable connected, in use, to a proximal end of the handle via electrical cabling extending through the handle.

The electrode sheath 20 is manufactured in accordance with the Applicant's above referenced International Patent Application No. PCT/AU01/01339. As indicated, the contents of that international patent application are incorporated in this specification by reference. The manufacturing method results in an unimpeded lumen of the electrode sheath 20. A steering device in the form of a steering shaft 36 (FIGS. 12 and 13) is received in the lumen of the electrode sheath 20.

The steering shaft 36 is of the type described in the Applicant's co-pending International Application No. PCT/AU2005/000216 dated 18 Feb. 2005 entitled “A steerable catheter”. The contents of that international application are incorporated in this specification by reference. Thus, the steering shaft 36 includes a tubular member in which an actuator is slidably received.

As described in that international application, the tubular member and the actuator of the steering shaft 36 are, respectively, fastened to relatively slidable portions of the handle for facilitating steering of the distal end of the electrode sheath.

The unit 10 includes a guide formation in the form of a guide tube 38. The steering shaft 36 passes through a passage defined by the guide tube 38.

In use, in this embodiment of the invention, the connector 12 is connected to the distal end of the handle of the catheter 16 with the steering shaft 36 passing through the guide tube 38 and being secured to the handle of the catheter 16.

The electrode sheath 20 is connected to the support 24 of the housing 22 with electrode conductors of the electrode sheath 20 being connected to the PCB 32.

To steer the catheter 16 to a site to be treated in a patient's body, the electrode sheath 20 is inserted into an introducer. The introducer is inserted into the vascular system of the patient, for example, via the femoral vein. The introducer is steered to the desired site using the steering device 36 of the catheter 16. As the introducer is steered, it is constrained by, and within, the vasculature of the patient's body which causes friction. The pressure applied by the introducer can be monitored due to the relative displacement of the connector 12 and the housing 22.

In this embodiment, the housing 22 moves in the direction of arrow 40 when pressure is applied to the support 18 of the housing 22. Thus, a clinician can monitor the pressure applied by the distal end of the introducer while the introducer is being steered. To perform a procedure, the distal end of the electrode sheath 20 is extended from a distal end of the introducer so that an ablation electrode 42 (FIGS. 12 and 13) at the distal end of the electrode sheath 20 is exposed. The electrode 42 is brought into contact with tissue to be ablated and the clinician can monitor the pressure applied to the tissue by the electrode 42. Thus, the clinician can control the pressure, in so doing to inhibit perforating either the vascular system of the patient or the tissue at the site being treated, as the case may be.

In this embodiment, the housing 22 includes a longitudinally extending slot 44. A distal end of the connector 12 is visible through the slot 44 and the clinician can, by monitoring the position of the distal end of the connector 12 relative to the housing 22, determine the pressure being applied.

In addition, a measuring device in the form of a load cell 46 is provided in the housing 22. A proximal end of the spring 28 bears against the load cell 46 and the load cell 46 is responsive to forces exerted on the load cell 46 by the spring 28. The spring force of the spring 28 is known. Thus, the load cell 46 is able to be calibrated to minimise, if not altogether eradicate, the effects of friction between the introducer and the electrode sheath 20. In this regard, it will be appreciated that, to inhibit backflow of bodily fluids, the introducer has a septum through which the electrode sheath 20 passes which can create friction when the electrode sheath 20 moves relative to the introducer. Further, as the introducer is navigated through the vascular system, it curves and, due to the curves and contact with the walls of the vessels, there is further friction. By appropriately calibrating the load cell 46, the effects of this friction can be minimised.

The load cell 46 is connected to a discernible alarm (not shown). The discernible alarm is an audio and/or a visual enunciator to warn the clinician when an over pressure situation occurs.

It is to be noted that, when the housing 22 moves in the direction of the arrow 40, there is relative displacement between the steering shaft 36 and the electrode sheath 20. Thus, as shown in FIG. 12 of the drawings, the steering shaft 36 terminates short of the distal end of the electrode sheath 20 carrying the ablation electrode 42.

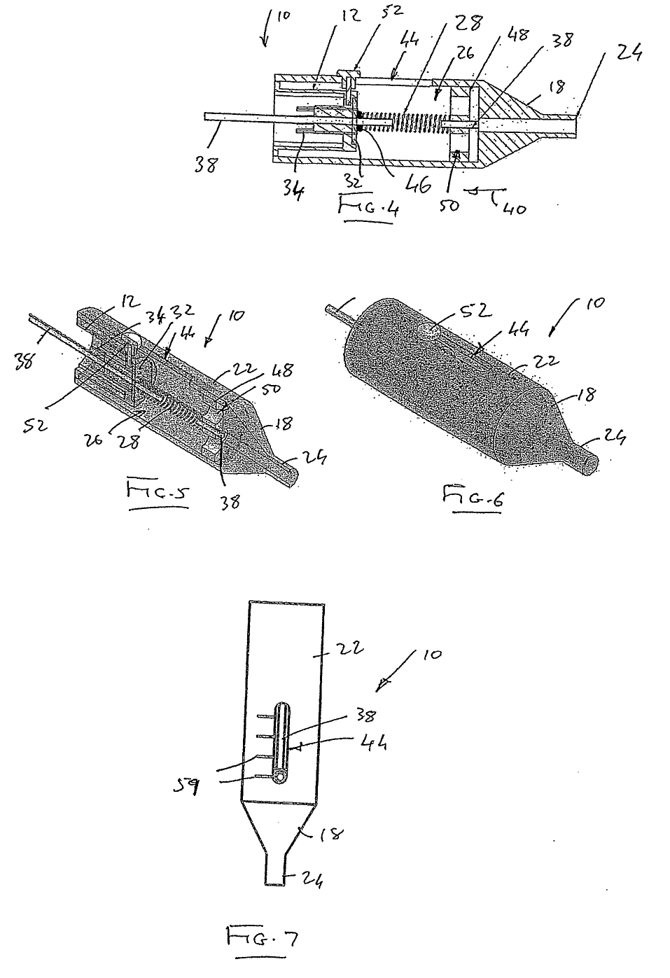

Referring now to FIGS. 4 to 6 of the drawings, a second embodiment of a pressure feedback unit 10 is illustrated. With reference to FIGS. 1 to 3 of the drawings, like reference numerals refer to like parts, unless otherwise specified.

In this embodiment, a proximal end of the electrode sheath 20 is attached to an attachment member in the form of an attachment plate 48. The attachment plate 48 is fixed in position in the interior 26 of the housing 22. The attachment plate 48 has a plurality of openings 50 through it. The electrode conductors of the electrode sheath 20 pass through these openings 50 to be connected to the PCB 32.

Also, instead of the clinician relying on the relative position of the connector 12 in the slot 44, an indicator 52 is arranged in the slot 44. When the housing 22 moves in the direction of the arrow 40, the indicator 52 slides along the slot 44 in the opposite direction providing a visual indication to the clinician of the pressure being applied.

The indicator 52 further acts as a thumb push which facilitates interconnection of the connector 12 and the distal end of the handle of the catheter 16.

In use, this embodiment of the unit 10 operates in substantially the same manner as described above with reference to the unit 10 of FIGS. 1 to 3 of the drawings.

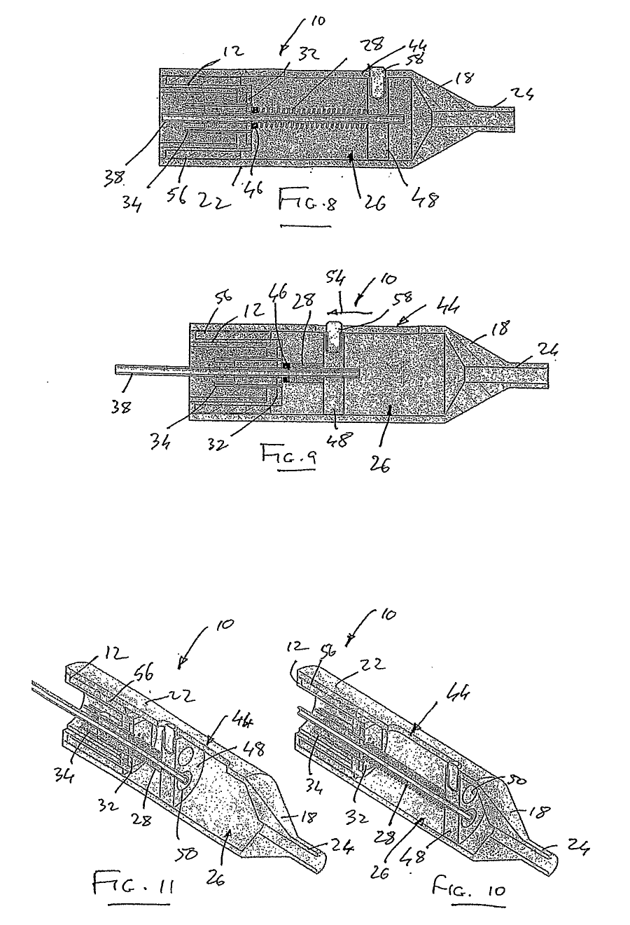

In FIGS. 7 to 10 of the drawings, yet a further embodiment of a pressure feedback unit 10 is illustrated. Once again, with reference to the previous drawings, like reference numerals refer to like parts, unless otherwise specified.

In this embodiment, the connector 12 is fixedly mounted in the proximal end of the housing 22. The connector 12 is held in position by an annular retaining member 56. Conversely, the attachment plate 48 is slidably displaceable, in a longitudinal direction, in the interior 26 of the housing 22. The guide tube 38 is fixedly secured to the attachment plate 48 and moves together with the attachment plate 48.

In this embodiment, when pressure is applied to the distal end of the electrode sheath 20 of the catheter 16, the attachment plate 48 moves in the direction of the arrow 54 (FIG. 9) against the action of the coil spring 28 thereby compressing the coil spring 28.

It is to be noted that the electrode conductors (not shown) from the electrode sheath 20 are coiled about the coil spring 28, both in this embodiment and in the previous embodiments, thereby permitting relative movement of the electrode sheath 20 and the steering shaft 36.

Further, in this embodiment of the invention, instead of the indicator 52, an indicator 58 is arranged in the slot which gives an indication of the applied pressure. Markings or gradations 59 are applied to the housing 22 alongside the slot 44. These markings 59 are indicative of the pressure being applied. If desired, similar markings may also be applied to the housing 22 of the unit 10 of each of the previous embodiments.

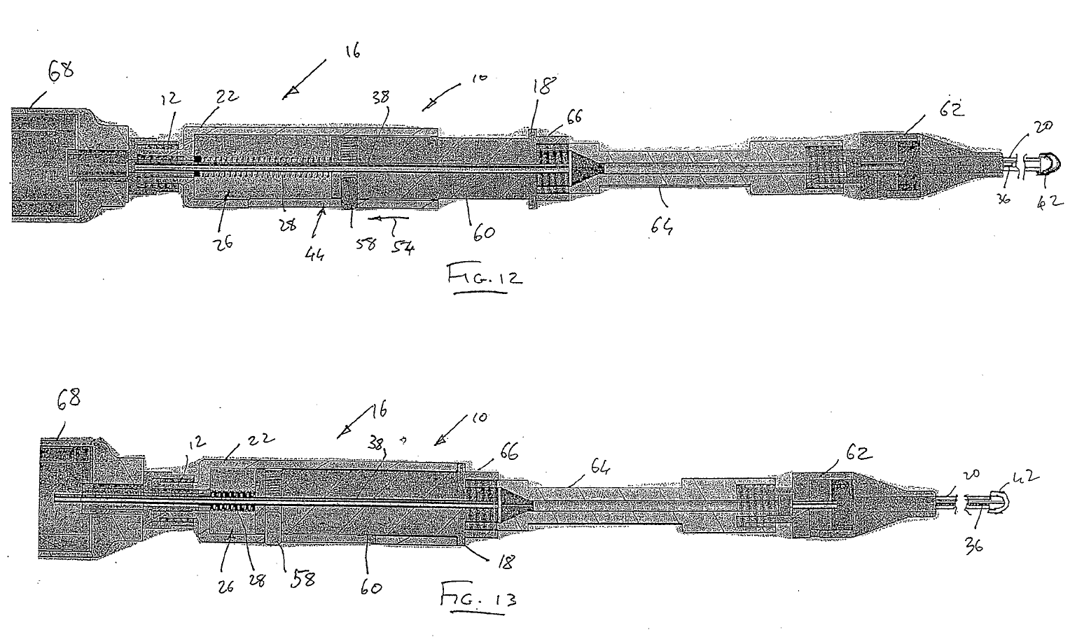

Once again, in this embodiment, the operation of the unit 10 is substantially the same as that described above. When no pressure is applied, the spring 28 is relaxed as shown in FIGS. 8 and 10 of the drawings and, when maximum pressure is applied, the spring 28 is compressed as shown in FIGS. 9 and 11 of the drawings.

Referring finally to FIGS. 12 and 13 of the drawings, yet a further embodiment of the unit 10 is shown. With reference to the previous drawings, like reference numerals refer to like parts, unless otherwise specified.

In this embodiment, the unit 10 operates in a piston-cylinder manner. The housing 22 acts as a cylinder and the connector 12 is mounted externally of the housing 22 at a proximal end of the housing 22. The support 18 for the electrode sheath 20 is in the form of a piston-like element 60 slidably received in the interior 26 of the housing 22 against which the coil spring 28 acts. In the illustrated embodiment, instead of the electrode sheath 20 being connected directly to the piston-like element 60 via an electrode sheath mount 62, a Y-connector 64 is interposed between the mount 62 and a distal end 66 of the piston-like element 60. A source of irrigation fluid to be supplied to a distal end of the electrode sheath 20 is connectable to the Y-connector 64. It will, however, be appreciated that, instead of the Y-connector 64 being interposed between the piston-like element 60 and the mount 62, the mount 62 could be connected directly to the distal end 66 of the piston-like element 60 or may, instead, be integrally formed with the distal end 66 of the piston-like element 60 as a one-piece unit.

In this embodiment, instead of the electrode conductors (not shown) of the electrode sheath 20 extending through the interior 26 of the housing 22, the conductors exit through the mount 62 and are arranged externally of the housing 22 and a handle 68 of the catheter 16.

The operation of this embodiment of the invention is the same as that described above with reference to FIGS. 8 to 11 of the drawings.

In yet a further embodiment, there may be very little, if any relative displacement between the connector and the support of the unit. A pressure transducer such as the load cell is interposed between the connector and the support with the connector and the support bearing on opposed sides of the load cell. In this embodiment, as there is pressure applied, either the connector or the support exerts a force on the load cell. The load cell emits a signal representative of the pressure applied. The pressure applied may be displayed or enunciated in a suitable manner such as an audio/visual display or alarm. Once again, the effects of system friction may be minimised by suitably calibrating the load cell.

It is therefore an advantage of the invention that a pressure feedback unit for a catheter is provided which enables a clinician readily to assess the pressure being applied both when the catheter is being steered via the introducer through the vascular system of the patient and when the ablation electrode at a distal end of the catheter is being urged into contact with tissue at a site of the patient's body to be treated. This reduces the risk of injury to the patient's body due to perforation of the vascular system or the tissue being treated.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A pressure feedback unit for a catheter, the unit comprising
a support for supporting a proximal end of an electrode sheath of the catheter;
a connector for connection to a component of the catheter, the connector and the support being displaceable relative to each other; and
an urging element interposed between the support and the connector for urging the support and the connector to a rest condition relative to each other.

2. The unit of claim 1 which includes a housing carrying the connector.

3. The unit of claim 2 in which the connector is supported at a proximal end of the housing and a distal end of the housing defines the support for the electrode sheath.

4. The unit of claim 3 in which the connector is displaceably arranged relative to the housing.

5. The unit of claim 2 in which the connector is fixed to a proximal end of the housing and the support comprises an attachment element displaceably arranged relative to the housing.

6. The unit of claim 1 in which the urging element is a coil spring.

7. The unit of claim 1 which includes a guide formation co-axially arranged with respect to the support and the connector, the guide formation defining a passage through which a steering device of the catheter passes, in use.

8. The unit of claim 1 in which the connector defines a receiving formation for receiving a part of the component.

9. The unit of claim 8 in which the connector includes an electrical connector for electrical connection between electrode conductors of the electrode sheath and electrical members associated with the component.

10. The unit of claim 1 which includes an indicator for indicating applied pressure.

11. The unit of claim 1 which includes a measuring device for measuring applied pressure, the measuring device being responsive to the urging element.

12. The unit of claim 11 which includes a discernible alarm connected to the measuring device.

13. A pressure feedback unit for a catheter, the unit comprising
a support for supporting a proximal end of an electrode sheath of the catheter;
a connector for connection to a component of the catheter; and
a pressure transducer interposed between the support and the connector, a force applied to the transducer by one of the connector and the support being monitored by the transducer to give an indication of pressure applied.
